(19) United States
(12) Patent Application Publication
(10) Pub. No.: US 2026/0041919 A1
(43) Pub. Date: Feb. 12, 2026

(54) SYSTEM INCLUDING ELECTROCEUTICAL FOR NERVE STIMULATION AND METHOD OF OPERATING THE SYSTEM

(71) Applicant: ENERGY MINING Co., Ltd., Suwon-si (KR)

(72) Inventors: Hyunmoon PARK, Gunpo-si (KR); Changheon LEE, Suwon-si (KR); Jangmook JEONG, Suwon-si (KR)

(73) Assignee: ENERGY MINING Co., Ltd., Suwon-si (KR)

(21) Appl. No.: 18/964,289

(22) Filed: Nov. 29, 2024

(30) Foreign Application Priority Data
Aug. 9, 2024 (KR) 10-2024-0106543
Aug. 9, 2024 (KR) 10-2024-0106546
Aug. 26, 2024 (KR) 10-2024-0114225

(51) Int. Cl. A61N 1/372; A61N 1/05; A61N 1/36; A61N 1/378

(52) U.S. Cl. CPC A61N 1/37229; A61N 1/0556; A61N 1/36175; A61N 1/36189; A61N 1/3787

(57) ABSTRACT

The present disclosure may include an electroceutical configured to sense a signal generated from a nerve and provide stimulation to the nerve, and an electronic device configured to control the electroceutical. The present disclosure relates to a system having an electroceutical for nerve stimulation, the electroceutical including a plurality of lead wires disposed to surround the nerve, a header connector including a plurality of pins connected to the plurality of lead wires; and a microcontroller unit (MCU) configured to change a setting of the header connector based on a control signal received from the electronic device.

### CROSS-REFERENCE TO RELATED APPLICATION

This application claims benefit of priority to Korean Patent Application No. 10-2024-0114225 filed Aug. 26, 2024, Korean Patent Application No. 10-2024-0106543 filed Aug. 9, 2024, and Korean Patent Application No. 10-2024-0106546 filed Aug. 9, 2024, the entire contents of which are incorporated herein by reference.

### BACKGROUND OF THE DISCLOSURE

### Field of the Disclosure

The present disclosure relates to a system including an electroceutical for nerve stimulation and a method of operating the system.

### Background of the Related Art

With an increasing demand for maintaining a healthy life in aging societies, a demand for implantable medical devices has grown rapidly since 2010, especially in advanced countries. In particular, along with advancements in state-of-the-art IT technology, the implantable medical devices become smaller, exhibit high performance, and require low power. Owing to a size reduction to a few centimeters, the implantable medical devices are now referred to as electroceuticals.

Stimulation and sensing of electroceuticals may be described in two methods. A first method is a stimulation method through event-based sensing. A representative method of the stimulation method is a ‘pacing (stimulation)’ technique provided by a cardiac pacemaker during arrhythmia occurrence. A second method is a method of performing regular stimulation after periodic sensing, which is mainly used in nerve stimulation and brain stimulation. This method is achieved by coordinating timing of sensing and stimulation from a therapeutic viewpoint.

The stimulation method is mainly performed by stimulating a nerve through a nerve stimulator. Nerve stimulation may be adjusted by monitoring external devices such as electroencephalography (EEG), electrocardiography (ECG), and electromyography (EMG) on a stimulation site, a brain, or a heart. Additionally, nerve stimulation may be adjusted after monitoring using intraoperative neuro monitoring (IONM) during surgery or monitoring through imaging tools such as magnetic resonance imaging (MRI) after surgery. Additionally, an effect of stimulation is measured through pain assessment tools such as a visual analog scale (VAS).

However, although a nerve would have fixed characteristics during stimulation, since a resistance value of the nerve is continuously changed in units of tens of kilo-ohms depending on human biorhythms, accuracy of nerve stimulation by the nerve stimulator is unknown and may only be inferred indirectly. Therefore, a method of providing nerve stimulation for 5 minutes, and then, taking a rest for 30 minutes, or visually observing a particular event in a nerve, or checking nerve stimulation through direct stimulation by a user is generally used, rather than providing continuous nerve stimulation. Recently, a method of implanting an electroceutical as an auxiliary unit, and then, performing treatment using personalized small-sized ECG and EMG monitoring devices is used, particularly in leading countries, which also has limitations in treatment.

Since operation of an electroceutical relies on a battery to operate like in 1970s, and thus, a device needs to be replaced due to battery depletion. To minimize side effects of surgeries due to replacement of implantable medical devices and relevant economic and psychological burdens caused by the surgeries, technologies for recharging implantable devices have been researched and commercialized for over 30 years.

However, a radio frequency (RF) method which is most commonly used has limitations in use due to a depth within a body, limitations in a charging amount, and safety issues. Since 2010, various charging methods such as an infrared (IR) method, radio frequency identification (RFID), a piezoelectric method, an optical method, and a thermoelectric method have been proposed. However, due to various limitations such as a low output, lack of durability, depth limitations, and an issue of harmlessness to the body, RF-based charging technologies have been still used mainly for medical purposes for over a decade.

In the 2020s, research into implantable electroceuticals that do not need batteries or battery replacement has become more diverse. Recently, research into electroceuticals that do not need batteries or battery replacement is being actively conducted.

### SUMMARY OF THE DISCLOSURE

Therefore, the present disclosure has been made to solve the above-described problems, and it is an object of the present disclosure to provide an electroceutical configured to sense a signal generated from a nerve and provide stimulation to the nerve. The electroceutical may be powered through an ultrasound wave in a low-frequency ultrasound (LFU) range or a low-intensity pulsed ultrasound (LIPUS) range based on an integrated triboelectric power harvesting unit. The electroceutical may perform sensing operation and stimulating operation by changing a setting of a header connector based on a control signal received from an electronic device. The electroceutical in the present disclosure may sense (measure) an accurate nerve stimulation value from a nerve in response to electrical stimulation to provide the accurate nerve stimulation value needed for the nerve. In addition, the electroceutical may actively perform sensing (measurement) and stimulation according to a condition of a patient by freely providing stimulation and receiving functions of the header connector. However, this is only an example, and the scope of the present disclosure is not limited thereto.

To accomplish the above object, according to one aspect of the present disclosure, an electroceutical configured to sense a signal generating from a nerve and provide stimulation to the nerve; and an electronic device configured to control the electroceutical may be included. A system having an electroceutical for nerve stimulation is provided, the electroceutical including: a plurality of lead wires disposed to surround the nerve; a header connector including a plurality of pins connected to the plurality of lead wires; and a microcontroller unit (MCU) configured to change a setting of the header connector based on a control signal received from the electronic device.

According to one example, the plurality of pins may include: one ground (GND) pin; and a plurality of general-purpose input/output (GPIO) pins individually controlled by the MCU.

According to one example, the plurality of GPIO pins may be connected to different neural regions, respectively, and each of the plurality of GPIO pins may sense a signal generated from the nerve or outputs a pulse signal according to a control by the MCU to provide stimulation to the nerve.

According to one example, the MCU may include: a first analog-to-digital converter (ADC) module configured to monitor a battery of the electroceutical; a second ADC module configured to sense an analog signal generated from a nerve; and a pulse width modulation (PWM) module configured to provide stimulation to the nerve, wherein the PWM module includes a hardware timer instead of a hardware PWM module, and the MCU sets a state of the plurality of GPIO pin based on a timer interrupt of the hardware timer.

According to one example, the electroceutical may include: a housing including a plurality of regions; a triboelectric power harvesting unit configured to generate energy based on an ultrasound wave provided from outside of the electroceutical; a plurality of antennas operating in different frequency bands; and shielding metal configured to shield electromagnetic interference caused by at least one of the plurality of antennas. The electroceutical may receive a control signal from the electronic device through an antenna having an operating frequency of 2.4 GHz among the plurality of antennas. A frequency band of 2.4 GHz is only an example, and a lower frequency band of 1 GHz or a Sub-GHz frequency may be used.

According to one example, the electronic device may display an interface for controlling the electroceutical. The interface may include: an interactive component configured to set an operating mode of each of the plurality of GPIO pins, the operation mode including a sensing mode, a stimulation mode, and a disable mode; and a component module provided to each of the plurality of GPIO pins in response to the set operation mode.

According to one example, the component module provided in response to the operation mode set to the stimulation mode may include: a first component configured to set a center frequency of a pulse signal generated for nerve stimulation; and a second component configured to set a pulse width of the pulse signal.

According to one example, the component module provided in response to the operation mode set to the stimulation mode may include: a third component configured to set whether to apply frequency random modulation to the pulse signal and set a range of the frequency random modulation with respect to the pulse signal; and a fourth component configured to set a ratio between the center frequency and a variable frequency in which the frequency random modulation is applied to the pulse signal.

According to one example, the triboelectric power harvesting unit may include: a power generating unit including a first unit, a second unit, and a third unit; a first silicon layer arranged between an upper surface of the power generating unit and an inner surface of the electroceutical; a second silicon layer arranged on a lower surface of the power generating unit; and a device housing arranged to surround outer peripheries of the power generating unit, the first silicon layer, and the second silicon layer.

According to one example, the ultrasound waves provided from the outside may penetrate through a titanium region from the plurality of regions and the first silicon layer to be introduced into the power generating unit, be reflected within the power generating unit, and cause repetitive vibrations.

According to one example, the electroceutical may monitor a voltage of the battery through the first ADC module and transmit the monitored voltage to the electronic device. The electronic device may calculate a remaining capacity of the battery based on the received voltage, provide the remaining capacity through an interface, and display a charging alarm of the electroceutical when the remaining capacity is equal to or less than a threshold value.

In addition to those described above, other aspects, features and effects will become apparent from the following drawings, claims, and detailed descriptions of the present disclosure.

As described above, according to one embodiment of the present disclosure, since one GPIO pin (and a lead connected thereto) included a header connector may perform a sensing function as well as a stimulation function, there is no need to separately provide a lead for sensing or a lead for stimulation. Thus, complexity of a device in association with a header and feed-through may be reduced in an implantable device or an electroceutical device.

As described above, according to one embodiment of the present disclosure, a ground pin included in the header connector may be used to make correction even when a sensing region and a stimulation region are separate from each other. Additionally, the header connector may include a plurality of GPIO pins to be capable of providing stimulation to a plurality of neural regions.

As described above, according to one embodiment of the present disclosure, a hardware timer may be utilized to generate a pulse width modulation (PWM) signal instead of using separate dedicated PWM hardware. The present disclosure may provide a high degree of freedom for setting stimulation by allowing to directly control a GPIO pin based on a timer interrupt.

As described above, according to one embodiment of the present disclosure, neural signals for stimulation and responses according to stimulation may be sensed and a related database (DB) may be secured and coordinated. Accordingly, an effect of reducing unnecessary energy consumption may be obtained by providing stimulation only when a particular event occurs. In addition, when physiological signals and information about neural responses to stimulation are combined with each other using methods such as closed-loop nerve stimulation, precise values for nerve stimulation may be sensed, and thus, which accurate and immediate stimulation control may be performed. On the other hand, since stimulation is needed in a method of performing periodic stimulation after periodic sensing from a therapeutic perspective, providing periodical sensing and stimulation at a regular interval may help to overcome particular pathologies.

As described above, according to one embodiment of the present disclosure, a stimulation pattern may be adjusted according to a condition of a patient through sensing and stimulation. Thus, efficiency of treatment may be enhanced. This method is particularly useful in central, vagus, and peripheral nerve stimulation and brain stimulation, and enables personalized treatment through changes in the stimulation patterns.

However, the scope of the present disclosure is not limited by the effects described above.

### BRIEF DESCRIPTION OF THE DRAWINGS

The above and other objects, features and advantages of the present disclosure will be apparent from the following detailed description of the embodiments of the disclosure in conjunction with the accompanying drawings, in which:

FIGS. 1 and 2 are diagrams for explaining a system including an electroceutical;

FIGS. 3A and 3B are a front view and a rear view of the electroceutical;

FIG. 4 is a front cross-sectional view of the electroceutical;

FIGS. 5 to 7 illustrate an interface of an electronic device configured to control the electroceutical; and

FIGS. 8 and 9 are diagrams for explaining a triboelectric power harvesting unit.

### DETAILED DESCRIPTION OF THE PREFERRED EMBODIMENT

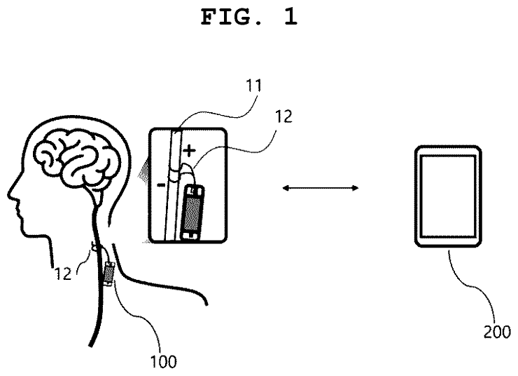

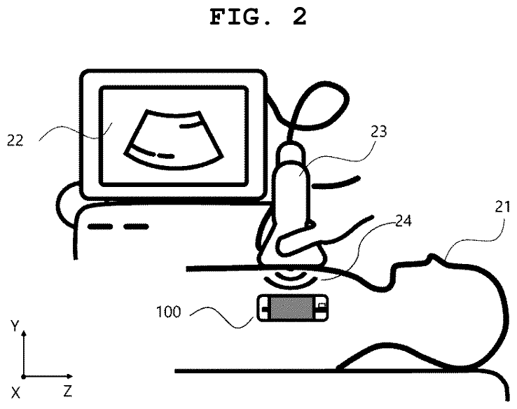

FIGS. 1 and 2 are diagrams for explaining a system including an electroceutical.

Referring to FIG. 1, an electroceutical 100 and an electronic device 200 are shown. The electroceutical 100 (e.g., an electronic device) may refer to a medical device implanted into a particular portion inside a body to stimulate a nerve 11 or sense biometric data. The electroceutical 100 may transmit the sensed biometric data to the electronic device 200, and receive a control command (e.g., a sensing command or a stimulation command) from the electronic device 200. That is, the electronic device 200 may be a device configured to control the electroceutical 100.

The electroceutical 100 is an implantable medical device and may be located at a depth of 2 cm or more inside the body. The electroceutical 100 is located deep in the body to be protected from an external impact or an environmental change.

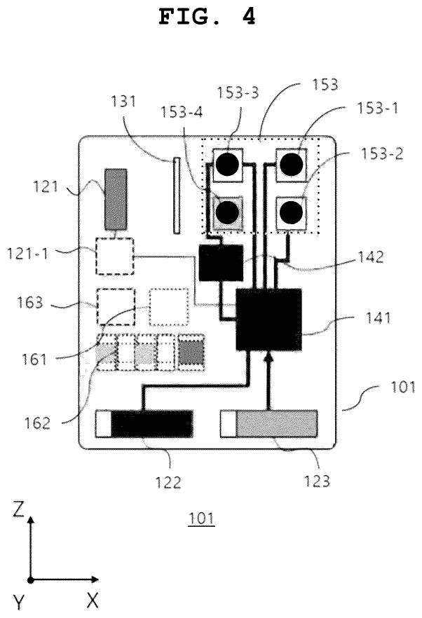

The electroceutical 100 may include a plurality of lead wires 12 (e.g., cuff leads) disposed to surround the nerve 11. The electroceutical 100 may control or improve a function of each organ through an electric signal provided through the lead wires 12. The electroceutical 100 may include a header connector (e.g., a header connector 153 of FIG. 4) including a plurality of pins connected to the plurality of lead wires 12. A setting of the header connector 153 may be changed by a microcontroller unit (MCU) (e.g., an MCU 141 of FIG. 4) based on a control signal received from the electronic device 200.

Referring to FIG. 2, a schematic diagram related to charging of the electroceutical 100 is shown. The electroceutical 100 may include a battery therein for long-term operation. However, the electroceutical 100 is a medical device implanted into a body of a person 21, and thus, it may not be easy to replace a battery. Therefore, the electroceutical 100 inside the body may charge a battery based on an ultrasound wave 24 provided from outside of the body.

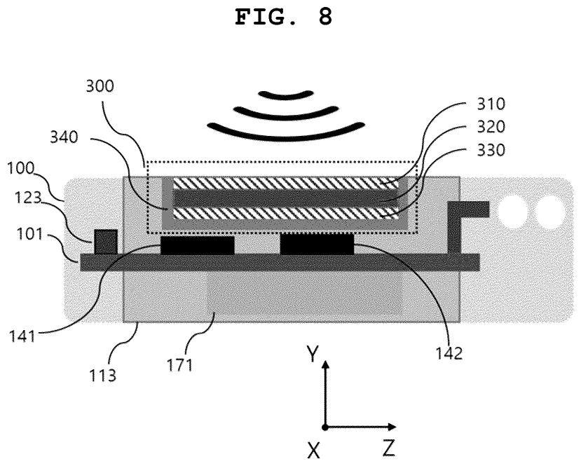

First, an ultrasound monitoring device 22 may be utilized to identify a position of the electroceutical 100 implanted into the body of the person 21. When the position of the electroceutical 100 is identified through the ultrasound monitoring device 22, the ultrasound wave 24 may be provided into the body through an ultrasound probe 23 (e.g., an ultrasound generator). The ultrasound wave 24 may penetrate through the body and be introduced into the electroceutical 100. The ultrasound wave 24 introduced into the electroceutical 100 may vibrate a triboelectric power harvesting unit (e.g., a triboelectric power harvesting unit 300 of FIG. 8) of the electroceutical 100. The vibration may generate electric charges within the triboelectric power harvesting unit, and the electroceutical 100 may be charged through the generated electric charges.

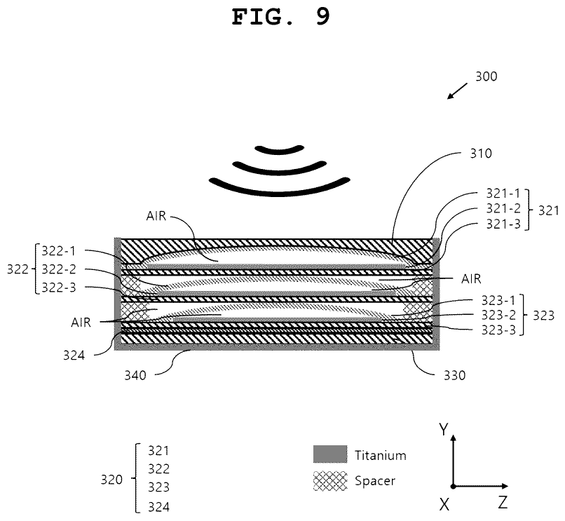

The electroceutical 100 with an approximately rectangular shape may be located such that two widest surfaces (e.g., surfaces parallel to an XZ plane) are parallel with an elongation direction (e.g., a Z-axis direction) of the person 21. Among the two widest surfaces, a surface located at a shallowest portion of the body may be defined as a front surface, and a surface located at a deepest portion may be defined as a rear surface. The ultrasound wave 24 may penetrate through the body to be introduced into the electroceutical 100 through the front surface of the electroceutical 100. Charging of the electroceutical 100 will be described in detail with reference to FIGS. 8 and 9.

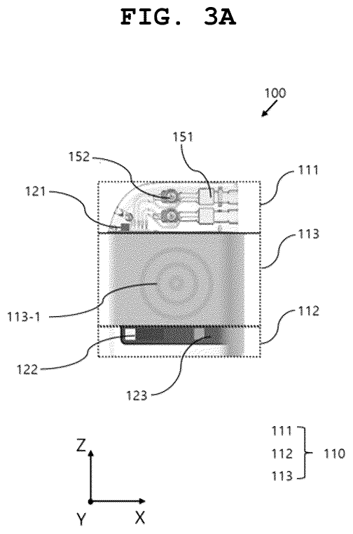

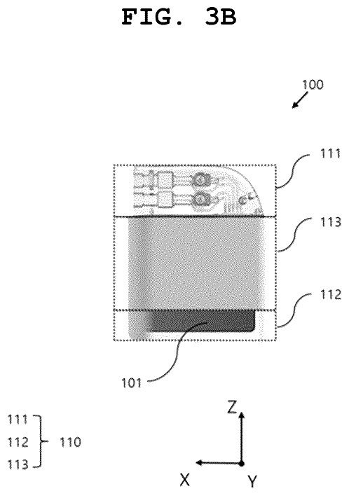

FIGS. 3A and 3B are a front view and a rear view of the electroceutical. FIG. 4 is a front cross-sectional view of the electroceutical.

Referring to FIG. 3A, a front view of the electroceutical 100 is shown, and referring to FIG. 3B, a rear view of the electroceutical 100 is shown.

The electroceutical 100 may include a housing 110 including a plurality of regions. The housing 110 may include a first silicon region 111, a second silicon region 112, and a titanium region 113.

The first silicon region 111 may be provided to cover at least a portion of a first antenna 121. The second silicon region 112 may be provided to cover a second antenna 122 and a third antenna 123. A plurality of the first to third antennas 121 to 123 operating in different frequency bands may be arranged on one surface of a printed circuit board (PCB) substrate 101.

A wireless communication signal may be transmitted or received through the first silicon region 111 and the second silicon region 112. At this time, the wireless communication signal may be attenuated by silicon. For example, in such a case that silicon having a Shore hardness of 60 A is included in the first silicon region 111 and the second silicon region 112, when a dielectric loss aϵᵣ is 3.7, a magnetic permeability is 4π×10⁻⁷ H/m, a frequency of the wireless communication signal is 2.4 GHz, and a thickness of the silicon is 5 to 15 mm, an attenuation α of the wireless communication signal may be approximately 0.00026 dB. That is, signal attenuation by the first silicon region 111 and the second silicon region 112 may be insignificant. The first silicon region 111 and the second silicon region 112 may preferably include silicon having a Shore hardness of 70 A or greater. When a Shore hardness of silicon is increased, transceiving sensitivity of a wireless communication signal may be increased.

Additionally, the first silicon region 111 may cover a lead connector 151 and a screw 152. The lead connector 151 may be connected to cuff leads (e.g., the cuff leads 12 of FIG. 2). The electroceutical 100 may sense an analog signal of a nerve or provide pulse stimulation to the nerve through the cuff leads connected to the lead connector 151. The screw 152 may be configured to connect the lead connector 151 to a header connector (e.g., the header connector 153 of FIG. 4).

The titanium region 113 may be located between the first silicon region 111 and the second silicon region 112. The titanium region 113 may be provided to cover the PCB substrate 101 and components placed on the PCB substrate 101. The titanium region 113 may include an ultrasound charging surface 113-1, and an ultrasound wave may be introduced into the electroceutical 100 through the ultrasound charging surface 113-1. Based on the ultrasound wave introduced into the electroceutical 100, a triboelectric power harvesting unit (e.g., the triboelectric power harvesting unit 300 of FIG. 8) may generate energy, and the generated energy may be used to operate the electroceutical 100. The triboelectric power harvesting unit is attached to an inner surface of the titanium region 113 to efficiently receive the ultrasound wave.

Referring to FIG. 4, components arranged on the PCB substrate 101 may be identified. The plurality of first to third antennas 121 to 123 operating in different frequency bands may be arranged on the PCB substrate 101.

In a communications field, a method of using a frequency band of 400 MHz for a wake-up purpose and a frequency band of 2.4 GHz for a data communication purpose is being commercialized, instead of a method of using a frequency band of 2.4 GHz for a wake-up purpose and a frequency band of 400 MHz for a medical implant communication service (MICS). This is because Bluetooth communication in the 2.4 GHz frequency band may provide sufficient low power property, security, and reliability thanks to advancements in short-range communication technology, low-power communication semiconductor technology, and high reliability secured through process advancement.

However, the use of the frequency band of 2.4 GHz for data communication may improve directivity of a data communication signal, but may cause problems such as a decrease in transmittance of the data communication signal and occurrence of environmental noise interference. To solve these problems, there is a need for a specific method of arranging and controlling an antenna.

Power for the electroceutical 100 may be controlled through the first antenna 121 operating in a near-field communication (NFC) band. That is, the electroceutical 100 may receive a power control signal from outside through the first antenna 121 and control power through an NFC module 121-1 (e.g., turning on/off). The power control signal may be received from an electronic device (e.g., the electronic device 200 of FIG. 1) or received from another external terminal.

The first antenna 121 may be positioned adjacent to the header connector 153 compared to other antennas such as the second and third antennas 122 and 123. Since the first antenna 121 is configured to transceive a power control signal, relatively low reliability is needed, and since the first antenna 121 functions for inductance, an influence exerted on the header connector 153 may be small. However, even when the influence is small, noise may be present. Thus, shielding metal 131 (e.g., mu-metal including nickel, iron, copper, and/or molybdenum) may be arranged. The shielding metal 131 may extend in a longitudinal direction (e.g., a Z-axis direction) of the first antenna 121 and arranged between the first antenna 121 and the header connector 153. Additionally, to reduce noise, a capacitor of approximately 0.1 μF to 1 μF may be arranged between a power rail and ground by using a decoupling method.

In addition, the first antenna 121 uses a short-range communication band to control power of the electroceutical 100, rather than utilizing a short-range communication band (e.g., an NFC band or a radio frequency identification (RFID) band) to charge the electroceutical 100. Thus, the first antenna 121 may have a small size compared to other antennas.

The second antenna 122 may have an operating frequency of 2.4 GHz. The second antenna 122 may transmit sensed biometric information to outside of the electroceutical 100 (e.g., the electronic device 200), and receive a control signal of the electroceutical 100 provided from the outside (e.g., the electronic device 200).

The third antenna 123 may have an operating frequency of 400 or 900 MHz. The third antenna 123 may be configured for a medical implant communication service (MICS). In detail, the third antenna 123 may be configured for an emergency control/call according to safety.

Frequency bands allocated for emergency control functions vary depending on countries, but a frequency band of 402 to 405 MHz at which highest body transmission is shown is allocated in most countries. This frequency range is intended for use in communication services between implantable medical devices, and thus, may be used for devices implanted into a body for medical purposes (Class III) (e.g., the electroceutical 100) Such implantable medical devices may use this frequency band to detect abnormalities present therein or to urgently provide control from outside. Due to high body transmissibility, the frequency band of 402 to 405 MHz is highly suitable for communications for medical use, and may perform an important function for increasing reliability and safety of the implantable medical devices in emergency situations despite restrictions in use of frequencies.

A MICS controls an electroceutical by employing an antenna device and components using frequencies that comply with national regulations. For example, when an electroceutical is urgently turned off via the MICS, a MICS-based RF chip needs to periodically wake up and scan an RF channel to turn the electroceutical on. However, periodic channel scanning consumes a lot of battery, and thus, may not be appropriate. Accordingly, power needs to be turned on using a method in which a battery is not consumed.

NFC may provide power using external radiation energy based on self-induction, and when used simply to perform a power control function, the NFC may be utilized even deep in a human body. Instead of NFC using a frequency of 13.56 MHz, a lower frequency band (e.g., 125 to 134 kHz) may also be used to perform simple power control.

When power is controlled through the NFC module 121-1, periodic energy consumption for MICS-based power control may not be needed. Power control using a general electromagnetic sensor may minimize a malfunction problem and reduce separate power consumption for a MICS-RF connection. Thus, this may help to operate the electroceutical 100 with a limited battery use.

The first antenna 121 connected to the NFC module 121-1 functions as a receiver (Rx), and is converted from a power-off state into a power-on state. Thus, the receiver (e.g., the first antenna 121) may have characteristics of an inductor configured to store energy of a magnetic field. Energy needed for the NFC module 121-1 performing power control may be sufficiently covered by only minimum energy stored in the first antenna 121.

That is, since the NFC module 121-1 receives power from the first antenna 121, a separate power line may not be needed. When power is supplied to the NFC module 121-1, an identification (ID) of a signal may be checked and, in a case of matching the ID, a power control signal may be provided to the MCU 141.

The MCU 141 may be a system on chip (SoC). The MCU 141 may perform sensing and stimulation simultaneously. The MCU 141 may include a first analog-to-digital converter (ADC) module, a second ADC module, and a pulse width modulation (PWM) module.

The first ADC module may monitor a battery (e.g., a battery 171 of FIG. 8) (or a storage 162 of FIG. 4) of the electroceutical 100. The first ADC module may monitor a voltage of the battery or the storage. The MCU 141 may periodically or aperiodically (e.g., according to a command in a case of occurrence of an event or randomly) transmit the monitored voltage to an electronic device (e.g., the electronic device 200 of FIG. 1).

The second ADC module may sense an analog signal of 14 bits or more generated from a nerve with high resolution (e.g., conversion into a digital signal). The PWM module may be configured to provide pulses of 0.1 Hz to several hundred kHz to the nerve as a stimulation. The PWM module may include a hardware timer instead of a hardware PWM module. In detail, the MCU 141 may set a state of general-purpose input/output (GPIO) pins (e.g., GPIO pins 153-1 to 153-3) included in the head connector 153 based on a timer interrupt of the hardware timer. That is, the MCU 141 may change a setting of the GPIO pins (e.g., the GPIO pins 153-1 to 153-3) according to an operating mode (e.g., a sensing mode or a stimulation mode).

The header connector 153 may include a plurality of pins. The header connector 153 may include a plurality of the GPIO pins 151-1 to 151-3 and one ground (GND) pin 153-4.

The plurality of GPIO pins 151-1 to 151-3 may be individually controlled by the MCU 141. The plurality of GPIO pins 151-1 to 151-3 may be connected to different neural regions, respectively. Each of the plurality of GPIO pins 151-1 to 151-3 may sense a signal generated from a nerve or output a pulse signal according to a control by the MCU 141 to provide a stimulation to the nerve.

The one GND pin 153-4 may be connected to ground to minimize an unexpected error (e.g., a sensing error or a stimulation error).

An operational (OP)-amplifier (AMP) 142 may be connected to the head connector 153. The OP-AMP 142 may be used to amplify or reduce a sensing signal or a provided stimulus (e.g., an electrical signal).

An alternating current (AC)-direct current (DC) converter 161 may convert AC triboelectricity generated by a triboelectric power harvesting unit (e.g., the triboelectric power harvesting unit 300 of FIG. 8) into DC triboelectricity.

A storage 162 may be a place for storing energy converted into DC (e.g., triboelectricity). The storage 162 may be implemented as a multi-stage capacitor (e.g., a system in which low resistance capacitors or supercapacitors of several hundred μF or several mF are connected to each other in series or parallel). The storage 162 may store energy in a wide frequency band (e.g., 0.2 to 200 kHz with a center frequency of 20 kHz). The storage 162 configured to store energy may also be provided to implement the electroceutical 100 free of a battery.

A power management integrated circuit (PMIC) 163 may be an integrated circuit configured to manage power for the electroceutical 100. The PMIC 163 may distribute power to elements included in the electroceutical 100. For example, the PMIC 163 may monitor a voltage to supply power to the MCU 141 or supply power to a battery (e.g., the battery 171 of FIG. 8) to charge the battery.

A battery management integrated circuit (BMIC) may be an integrated circuit configured to monitor and manage a state of the battery. The BMIC may manage charging and discharging of the battery. The BMIC and the battery may be placed on a rear surface (e.g., another surface) of the PCB substrate 101, or may not be included in the electroceutical 100. The BMIC may have a form integrated into a PMIC, but is not limited thereto.

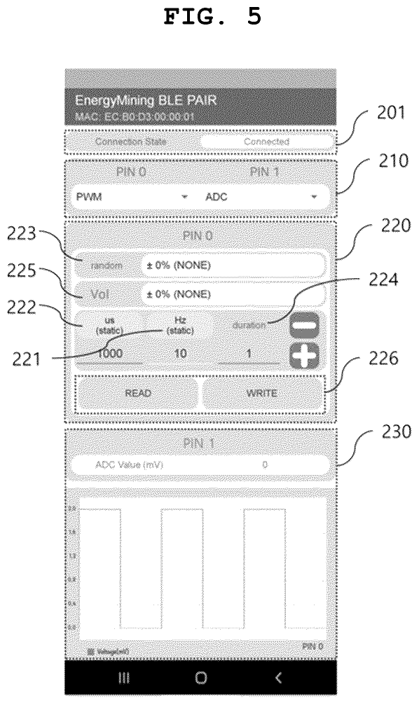

FIGS. 5 to 7 illustrate an interface of an electronic device configured to control the electroceutical.

First, as described above, an electroceutical (e.g., the electroceutical 100 of FIG. 1) may generate energy based on an ultrasound wave introduced therein. Generation of energy may be performed by a triboelectric power harvesting unit (e.g., the triboelectric power harvesting unit 300 of FIG. 8), and the generated energy may be used to operate the electroceutical 100. To block noise generated during the generation of the energy by the triboelectric power harvesting unit 300 and noise generated during a communication process, shielding metal (e.g., mu-metal) may be provided inside the electroceutical 100. That is, the electroceutical 100 may be designed to minimize an influence of, on other circuits, noise generated during ultrasound-based charging.

The electroceutical 100 is implanted into a body as described above with reference to FIG. 1. An implantation location is not limited and the electroceutical 100 may be implanted into various regions. A plurality of lead wires (e.g., the lead wires of FIG. 1) included in the electroceutical 100 may be disposed to surround a nerve in the body. The electroceutical 100 may sense a signal of the nerve through the lead wires and provide a stimulation to the nerve.

During ultrasound charging of the electroceutical 100, noise caused by an ultrasound wave may be incorrectly sensed as a signal of the nerve by the electroceutical 100. To solve this problem, the triboelectric power harvesting unit 300 may include shielding metal made of mu-metal as a device housing (e.g., a device housing 340). In addition, to solve this problem, an operation mode of the electroceutical 100 may be subdivided (e.g., into an ultrasound charging mode and an ultrasound diagnosis (MRI) mode) and managed.

As described above with reference to FIG. 4, the header connector 153 of the electroceutical 100 may include three GPIO pins 153-1 to 153-3. Each of the three GPIO pins 153-1 to 153-3 may be freely set to either stimulation or sensing.

In the related art, since a sensing lead is separate from a stimulation lead, there is a problem in that an electroceutical has a large size due to an increase in hardware complexity and an increase in a number of leads. In this case, an exclusive use may be ensured, but a structure of a connector header is complicated, thus leading to a structure having a large electroceutical. The electroceutical 100 may have the three GPIO pins 153-1 to 153-3 that may be freely set for stimulation and sensing, and thus, complexity of a device may be resolved and a size may be reduced.

In the related art, stimulation is basically performed based on sensing to stimulate a nerve, and an intensity and an interval of the stimulation are limited to a doctor with a medical diagnostic authority. Sensing methods include a method of observing a biomedical electrocardiogram (ECG)/electroencephalogram (EEG) and a method of monitoring cardiac information. In these methods, muscle and nerve movement by nerve stimulation is indirectly monitored in ECG/EEG from outside. The monitoring of cardiac information is also an indirect method for checking side effects of a stimulation or related responses rather than for therapeutic purposes.

In the electroceutical 100, the three GPIO pins 153-1 to 153-3 other than the one GND pin 153-4 may be freely set, and the one GND pin 153-4 may be used as ground for correction during stimulation or sensing. When a sensing region and a stimulation region are close to each other in a nerve, use of a housing of the electroceutical 100 made of titanium as ground may not have a significant influence on stimulation and sensing. However, when the stimulation region is apart from the sensing region by 10 cm or more, the influence may increase to a level that cannot be ignored. Therefore, the electroceutical 100 may be provided to allow correction through the GND pin 153-4 even when the sensing region is partially separate from the stimulation region. In addition, when a feedthrough is connected, a connection between the GND pin 153-4 and the GPIO pins 153-1 to 153-3 needs to be made. The electroceutical 100 may be designed to have a structure for making the connection.

The GPIO pins 153-1 to 153-3 of the electroceutical 100 may freely perform sensing (read) or stimulation (pacing) according to the setting, instead of fixedly performing the sensing or the stimulation. For example, one GPIO pin may be set to perform stimulation and two GPIO pins may be set to perform sensing. As another example, all three GPIO pins may be set to perform sensing to monitor a nerve with respect to external stimulation. The electroceutical 100 may secure a neural response threshold (NRT) through sensing, and thus, may detect an accurate response time (μs to ms) according to stimulation to a nerve and a voltage (mV to V) according to stimulation to a nerve.

Since the electroceutical 100 implements sensing and stimulation on a single pin under such an active control, consumption of invalid power due to a separate pin configuration may be minimized. The electroceutical 100 may efficiently stimulate a nerve with minimal power, and in a medical perspective, may achieve a maximum medical effect with a minimal stimulation.

Referring to FIG. 5, an example of an electroceutical control interface displayed on the electronic device 200 is shown. The electroceutical control interface may include an interactive component 210 and component modules 220 and 230.

First, the electroceutical control interface may include the component 201. The component 201 may indicate a connection state (e.g., connected) between the electroceutical 100 and the electronic device 200. The connection state may include connected, non-connected, and a low-power state (sleep). The connection state may be displayed through the component 201.

The interactive component 210 may be configured to set an operating mode of each of a plurality of GPIO pins. The three GPIO pins 153-1 to 153-3 may be displayed as PIN 0, PIN 1, and PIN 2 on the interactive component 210. Although only PIN 0 and PIN 1 are shown in FIG. 5, this is only an example for explanation, and the scope of the present disclosure is not limited to the illustration shown in FIG. 5.

Operation modes of the three GPIO pins 153-1 to 153-3 may include a sensing mode, a stimulation mode, and a disable mode. On the interactive component 210, the sensing mode may be displayed as ADC, the stimulation mode as PWM, and the disable mode as Disable. Referring to FIG. 5, PIN 0 corresponding to the GPIO pin 153-1 may be set to a present stimulation mode (e.g., displayed as PWM), and PIN 1 corresponding to the GPIO pin 153-2 may be set to a present sensing mode (e.g., displayed as ADC).

The component modules 220 and 230 may be provided to each GPIO pin in response to a set operation mode. The component module 220 may be provided with respect to PIN 0 corresponding to the GPIO pin 153-1. The component module 230 may be provided with respect to PIN 1 corresponding to the GPIO pin 153-2.

The component module 220 may display an element regarding the GPIO pin 153-1 (e.g., PIN 0) set to the stimulation mode. The component module 220 provided in response to setting of the stimulation mode may include components 221 to 226.

The first component 221 may be a component configured to set a center frequency of a pulse signal generated to stimulate a nerve. For example, when the center frequency is set to 10 Hz, the nerve may be stimulated by generating a pulse signal 10 times per second.

The second component 222 may be a component configured to set a pulse width of a pulse signal (e.g., pulse duration time). For example, when a pulse width is set to 1000 μs, a pulse signal lasts for 1000 μs once. A pulse width may be set in units of 25 μs, and a settable range may be determined in consideration of a preset center frequency.

The third component 223 may be a component configured to set whether to apply frequency random modulation to a pulse signal and set a range of the frequency random modulation with respect to the pulse signal. A learning effect of a nerve in a body may be beyond human imagination. When a nerve is continuously exposed to a pulse signal at a same interval, the nerve may ignore the pulse signal due to neural adaptation which is a phenomenon in which a nervous system perceives a repetitive stimulation as meaningless or sensory adaptation which is a phenomenon in which sensory receptors decrease sensitivity to continuous stimulation. Additionally, in consideration of a phenomenon of neural habituation or neural desensitization, randomness may be needed in a pulse signal applied as a stimulus to a nerve.

A frequency random modulation range may be set through the third component 223 based on %. For example, the frequency random modulation range may be any of 0%, 3%, or 5%. When a center frequency of a pulse signal is set to 1 Hz and a range of frequency random modulation is set to 3%, a pulse signal of 0.97 to 1.03 Hz may be randomly provided to a nerve.

The fourth component 224 may be a component configured to set a ratio between a center frequency and a variable frequency in which frequency random modulation is applied to a pulse signal. For example, in such a case that the fourth component 224 is set to 1, a center frequency of a pulse signal is set to 1 Hz, and frequency random modulation is applied, when the electroceutical 100 provides stimulation for 60 times per minute, a pulse signal may be generated only once in accordance with the center frequency, and generated for 59 times in accordance with the variable frequency.

The fifth component 225 may be a component configured to set a voltage value of a pulse signal. A voltage value of the pulse signal may be controlled in units of 0.1 V. With respect to vagus nerve stimulation (VNS), central nervous stimulation (CNS), and peripheral nerve stimulation (PNS), a resistance in a portion in which a medical procedure (an operation) has been performed once is not generally changed by 10% or more when a physical change except for a target disease is not present. Therefore, when a voltage of the pulse signal is decreased, current is resultantly decreased, and when a voltage is increased, current is resultantly decreased, thereby stimulating a corresponding nerve through constant power.

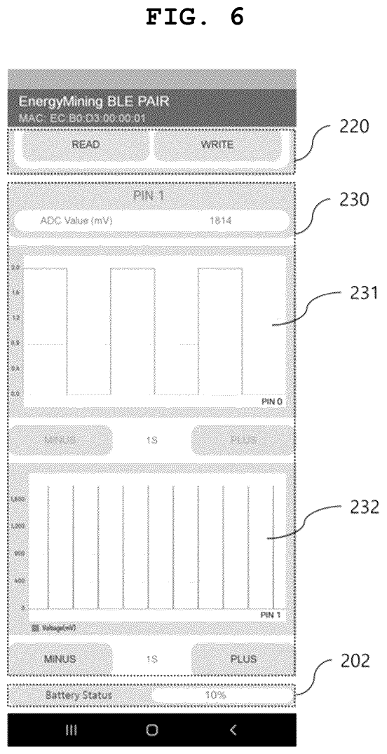

Referring to FIG. 6, another example of an electroceutical control interface displayed on an electronic device 200 is shown. The electroceutical control interface may include the component modules 220 and 230.

The component module 230 may display an element regarding the GPIO pin 153-2 (e.g., PIN 1) set to the sensing mode. The component module 220 provided in response to the setting of the sensing mode may include a plurality of graphs 231 and 232. The graph 231 may be a graph for stimulation. The graph 232 may be a graph for sensing.

Hereinafter, a sensing operation performed by the electroceutical 100 is described. A resolution of sensing may be defined as 12 bits by default. When general sensing is performed in addition to normal treatment (or diagnosis), a sampling rate and resolution (bit) may be lowered to reduce battery consumption. For example, by performing sensing at a sampling rate of 1 kHz and a resolution of 12 bits for diagnosis, values such as response time, a voltage, etc. of a nerve may be precisely monitored, and then, the sampling rate and the resolution of the sensing may be reduced. By setting a resolution and a sampling rate and defining a range, various stimuli and/or responses (e.g., responses to internal electrical stimulation, external electrical stimulation, and internal nerve stimulation) in various nerves (e.g., vagus nerves, central nerves, peripheral nerves, etc.) may be sensed in units of tens of PV to mV.

Generally, in a case of peripheral nerve stimulation (PNS), a voltage of several hundred mV flows through nerves for several hundred ms through a simple external stimulus. In this case, sufficient sensing may be performed with a resolution of 8 or 10 bits instead of 12 bits and a sampling rate set to 50 Hz.

However, in a case of central nervous stimulation (CNS) or vagus nerve stimulation (VNS), characteristics of nerves are diverse and response time is reduced to milliseconds. Thus, various options may be provided for treatment. A resolution may be set from among 12, 10, or 8 bits. A sampling rate may also be set from 10 Hz to 20 kHz in units of 10 Hz by actively utilizing a dynamic sampling rate control supported by analog-to-digital converter (ADC) hardware. For example, neural response time for VNS is 5 to 20 ms, and a sampling rate of 500 Hz or higher is needed to perform sensing at an interval of 20 ms. Depending on a resolution and a measurement setting range supported by the ADC hardware, sensing may be performed from 0.1 mV or 0.2 mV in units of 1 mV or 2.5 mV. Accordingly, the electroceutical 100 may sense nerve stimulation of 0 to 2 V and/or 0 to 1 V.

The electroceutical control interface may further include a component 202. The component 202 may indicate a remaining capacity (e.g., 10%) of a battery (e.g., the battery 171 of FIG. 8) (or the storage 162 of FIG. 4). The remaining capacity of the battery or the storage may be calculated based on a voltage monitored by the first ADC module of the electroceutical 100. The electronic device 200 may calculate a remaining capacity of the battery or the storage based on a voltage received from the electroceutical 100 and provide the remaining capacity (e.g., 10%) through the component 202. Additionally, when the remaining capacity is equal to or less than a threshold value (e.g., 10%), the electronic device 200 may display a charging alarm (not shown) of the electroceutical 100.

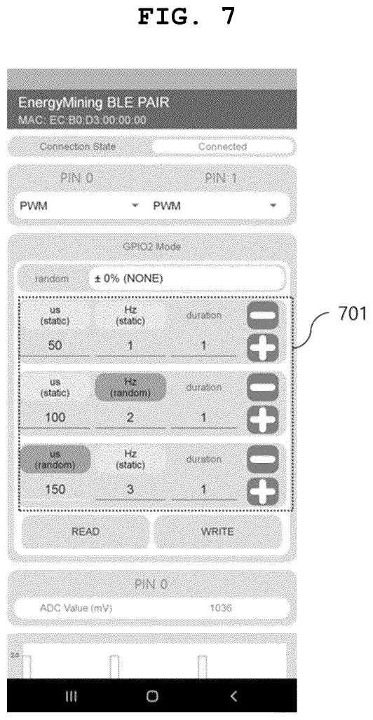

Referring to FIG. 7, another example of an electroceutical control interface displayed on an electronic device 200 is shown.

As described above, the electroceutical 100 may generate different pulse width modulation (PWM) signals using the plurality of GPIO pins 153-1 to 153-3, respectively, and based on this, provide a pulse stimulation to different nerve areas. A PWM-based stimulation method is based on direct current (DC) stimulation, but is not limited thereto. For example, a stimulus may be configured in various forms such as pulse waves, triangular waves, rectangular waves, complex waves, oscillatory waves, and high frequency waves. The electroceutical 100 may directly control the GPIO pins 153-1 to 153-3 through a timer interrupt instead of using separate dedicated PWM hardware to generate PWM signals.

Generally, an MCU uses dedicated PWM hardware to generate PWM signals. However, the electroceutical 100 generates a PWM signal by directly controlling the GPIO pins 153-1 to 153-3. In the electroceutical 100, states of the GPIO pins 153-1 to 153-3 may be changed at a regular interval using the timer interrupt (e.g.: 0 when high and 1 when low).

The electroceutical 100 may utilize the hardware timer in units of 2 μs. The hardware timer may generate a timer interrupt at an interval of 25 μs. By monitoring states of the GPIO pins 153-1 to 153-3 whenever the timer interrupt occurs, the states of the GPIO pins 153-1 to 153-3 may be changed in a case of timing matching. For example, to generate a pulse signal of 1 kHz at an interval of 1 ms, the electroceutical 100 may change the states of the GPIO pins 153-1 to 153-3 by generating an interrupt for 40 times in one period.

In this case, it may be difficult to generate a plurality of PWM signals. However, since the electroceutical 100 has three GPIO pins 153-1 to 153-3, generation of one PWM signal may be allocated to each of the GPIO pins 153-1 to 153-3 to perform individual control (see a reference numeral 701 in FIG. 7). A method of directly controlling GPIO pins consumes more power than a PWM control method. For example, the method of directly controlling GPIO pins may consume power up to 10 times more than that of the PWM hardware control method. However, the GPIO pins may be controlled sufficiently in a low frequency range. Since reliability may be secured and various modes may be provided In a low frequency range, a method of directly controlling a GPIO pin may have more advantages over a PWM hardware control method. Also, when low power property and various elements are utilized, loss caused by power consumption may be reduced compared to the PWM hardware control method. In addition, controlling is performed at a level of nA to μA in the PWM hardware control method, whereas in the method of directly controlling a GPIO pin, controlling is performed at a level of nA to tens of μAs. However, when a controlling method is set to be performed in multiple steps, controlling may be performed at a level of μA to several μAs in the method of directly controlling the GPIO pin.

As described above, the electroceutical 100 may stimulate a plurality of neural regions by directly controlling the plurality of GPIO pins 153-1 to 153-3 using a hardware timer. Accordingly, the electroceutical 100 may control stimulation with a high degree of freedom (e.g., a wide range of frequencies of pulse signals may be set, random variation of frequency may be performed). In the electroceutical 100, each of PIN 0, PIN 1, and PIN 2 may be freely set within a frequency of 1 Hz to 20 kHz (e.g., 1 Hz/2 kHz/300 Hz or 1 kHz/10 Hz/30 Hz, and setting to random may be also performed).

The electroceutical 100 capable of stimulating a plurality of nerves may be used for various neuropathies and may be adjusted by an engineer through a person with a diagnosis and prescription authority. The electroceutical 100 may perform nerve stimulation efficiently for a long time with only a small amount of battery. In addition, the electroceutical 100 may minimize a side effect through precise nerve stimulation. Further, the electroceutical 100 may provide stimulation to various regions such as peripheral nerves and central nerves as well as vagus nerves by changing only a lead wire. The electroceutical 100 may be very highly utilized. In addition, the electroceutical 100 provides not only stimulation to vagus nerves at an appropriate speed (e.g., a stimulus of 1 kHz or higher), but also provides stimulation in association with a voltage level and a current level. Therefore, the electroceutical 100 may provide stimulation at a voltage level of 1.2 to 2 V and a current level of Ma, and may efficiently provide stimulation to vagus nerves and peripheral nerves.

FIGS. 8 and 9 are diagrams for explaining a triboelectric power harvesting unit.

FIG. 8 is a right cross-sectional view of the electroceutical 100. Referring to FIG. 8, the electroceutical 100 (e.g., an electronic device) may include the triboelectric power harvesting unit 300 for charging. The triboelectric power harvesting unit 300 may be attached to a front surface (e.g., an inner surface of the titanium region 113 of the housing 110)) of the electroceutical 100 to effectively receive an ultrasound wave provided from outside of the electroceutical 100.

The triboelectric power harvesting unit 300 may include a first silicon layer 310, a power generating unit 320, a second silicon layer 330, and a device housing 340. The triboelectric power harvesting unit 300 may be designed to 1) maximize charging efficiency, and 2) minimize an influence of the triboelectric power harvesting unit 300 on operation of the electroceutical 100 (e.g., sensing, stimulation, and communication).

The triboelectric power harvesting unit 300 may be attached to the inner surface of the titanium region 113 and spaced apart from the PCB substrate 101.

The first silicon layer 310 may be disposed between on the inner surface (e.g., a front surface) of the titanium region 113 and an upper surface of the power generating unit 320. The first silicon layer 310 may minimize an air gap between an inner surface of the electroceutical 100 (e.g., an inner surface made of titanium) and the power generating unit 320. Since it is difficult for an ultrasound wave to penetrate through the air gap, the first silicon layer 310 may be provided to prevent the ultrasound wave from failing to reach the power generating unit 320 and being reflected on the air gap (approximately 99% reflected). The first silicon layer 310 may maximize charging efficiency of the electroceutical 100 by increasing transmission efficiency of the ultrasound wave.

The power generating unit 320 may generate electricity based on an ultrasound wave (e.g., an ultrasound wave having penetrated through the housing 110 of the electroceutical 100 and the first silicon layer 210) provided from outside (e.g., outside the electroceutical 100). The power generating unit 320 may generate triboelectricity as internal components disposed therein vibrate due to the ultrasound wave. A configuration and operation of the power generating unit 320 will be described in detail with reference to FIG. 9.

The second silicon layer 330 may be disposed on a lower surface of the power generating unit 320. The second silicon layer 330 may suppress physical vibrations in units of μm generated in the power generating unit 320 from being delivered to outside of the triboelectric power harvesting unit 300. The second silicon layer 330 may be provided to minimize an influence of the triboelectric power harvesting unit 300 on operation of the electroceutical 100 (e.g., sensing, stimulation, and communication).

The device housing 340 may be arranged to surround outer peripheries of the first silicon layer 310, the power generating unit 320, and the second silicon layer 330. The device housing 340 may be also provided to minimize an influence of the triboelectric power harvesting unit 300 on operation of the electroceutical 100 (e.g., sensing, stimulation, and communication). The device housing 340 may be disposed to surround surfaces of the triboelectric power harvesting unit 300 other than an upper surface thereof to shield electromagnetic interference between the triboelectric power harvesting unit 300 and the outside. The device housing 340 may be made of mu-metal including nickel, iron, copper, and/or molybdenum to shield electromagnetic interference.

The mu-metal may effectively shield a magnetic field (e.g., 40 to 60 dB) in a low frequency band (e.g., a frequency band below 10 kHz) generated inside the triboelectric power harvesting unit 300. The triboelectric power harvesting unit 300 may also generate a magnetic field of several tens of mV while producing AC triboelectricity. The generated magnetic field may function as noise in other circuits.

Accordingly, the device housing 340 may be provided to surround surfaces of the triboelectric power harvesting unit 300 other than an upper surface thereof to receive an ultrasound wave. The upper surface of the triboelectric power harvesting unit 300 may be attached to an inner surface of the electroceutical 100 (e.g., an inner surface of the titanium region 113) to package the triboelectric power harvesting unit 300. As a result, an operation error of the electroceutical 100 caused by the triboelectric power harvesting unit 300 may be minimized.

In addition, the mu-metal may effectively shield a magnetic field (e.g., a magnetic field introduced when magnetic resonance imaging (MRI) is performed on a body) introduced from outside of the triboelectric power harvesting unit 300.

FIG. 9 is a right side view of the triboelectric power harvesting unit 300.

Referring to FIG. 9, the right-side view of the triboelectric power harvesting unit 300 is shown. The triboelectric power harvesting unit 300 may include the first silicon layer 310, the power generating unit 320, the second silicon layer 330, and the device housing 340.

The power generating unit 320 may include a plurality of units 321 to 323 and a ceramic substrate 324. The plurality of units 321 to 323 may be stacked on the ceramic substrate 324.

The ceramic substrate 324 has a thermal conductivity of 200 W/mK which is 60 times higher than a thermal conductivity of a general PCB substrate (e.g., 0.4 W/mK), showing excellent heat dissipation performance. A thermal expansion coefficient of the ceramic substrate 324 is 7 ppm/° C. which is lower than a thermal expansion coefficient (e.g., 14 ppm/° C.) of a general PCB substrate (e.g., High Tg-PCB). The ceramic substrate 324 has a high temperature resistance of 800° C., which is much higher than a high temperature resistance (e.g., 185 to 220° C.) of a general PCB substrate (e.g., High Tg-PCB). Thus, the ceramic substrate 324 may withstand an extremely high temperature. In addition, the ceramic substrate 324 has a dielectric constant and a mechanical strength higher than those of a general PCB substrate (e.g., a flame retardant (FR)-4 PCB of 140 Mpa).

The plurality of units 321 to 323 stacked on the ceramic substrate 324 may include a first unit 321, a second unit 322, and a third unit 323.

The plurality of units 321 to 323 may include inductive bodies 321-1, 322-1, and 323-1 that vibrate according to ultrasound waves, electrified bodies 321-2, 322-2, and 323-2 that generate triboelectricity according to friction with an inductive body, and supports 321-3, 322-3, and 323-3 each supporting an electrified body, respectively.

The inductive bodies 321-1, 322-1, and 323-1 may include barium titanate (BaTiO₃). The inductive bodies 321-1, 322-1, and 323-1 may include thin films and vibrate. The inductive bodies 321-1, 322-1, and 323-1 may vibrate according to transmission of ultrasound waves. The inductive bodies 321-1, 322-1, and 323-1 may vibrate within the air layer AIR, and the inductive bodies 321-1, 322-1, and 323-1 may be provided to have a thickness of 15 μm or less to efficiently vibrate.

The electrified bodies 321-2, 322-2, and 323-2 may contain gold (Au). The electrified bodies 221-2, 222-2, and 223-2 may generate triboelectricity due to friction with a vibrating inductive body. The electrified bodies 321-2, 322-2, and 323-2 may contain not only gold (Au), but also a material in which frictional electricity (e.g. nickel, silver, etc.) may be easily generated.

The supports 321-3, 322-3, and 323-3 may support an electrified body. Spacers may be interposed between the supports 321-3, 322-3, and 323-3. Areas of respective units may be distinguished from each other by the spacers.

In the first unit 321 and the second unit 322, the supports 321-3 and 322-3 may include barium titanate, zirconia (ZrO₂), and/or alumina (Al₂O₃). Zirconia (ZrO₂) and alumina (Al₂O₃) may be materials with high reflectivity. Zirconia (ZrO₂) and alumina (Al₂O₃) may have a reflectivity 10 times higher than that of FR-4 including an epoxy resin and glass fiber.

For example, in the first unit 321, an ultrasound wave having penetrated through the inductive body 321-1, the air layer AIR, and the electrified body 321-2 may be reflected by the support 321-3 with high reflectivity. The reflected ultrasound wave may induce repetitive vibrations within the first unit 321. Repetitive vibrations may increase energy efficiency of the triboelectric power harvesting unit 300. That is, an ultrasound wave provided from outside may penetrate through the titanium region 113 and the first silicon layer 310 to be introduced into the power generating unit 320, and reflected in the power generating unit 320 to cause repetitive vibrations.

In the third unit 323, the support 323-3 may include a fluorine compound (e.g., perfluoroalkoxy (PFA), fluorinated tetrafluoroethylene (FTFE), perfluorodecanoic acid (PFDA), and polydiacetylene (PDA)). The fluorine compound constituting the support 323-3 may have a higher reflectivity than that of the zirconia (ZrO₂) and alumina (Al₂O₃) constituting the supports 321-3 and 322-3. Referring to Table 1, a transmittance of each material is shown (transmittance=1−reflectivity)

TABLE 1

| Transmittance | Al²O³ | BTO | ZrO² | FR-4 | PDA | PFA | PVDF | PFDA | PTFE |
|---|---|---|---|---|---|---|---|---|---|
| Alumina (Al²O³) | 0.074 | 0.068 | 0.066 | 0.016 | 0.011 | 0.010 | 0.015 | 0.010 | 0.010 |
| BTO (BaTiO³) | 0.068 | 0.062 | 0.061 | 0.015 | 0.010 | 0.009 | 0.014 | 0.009 | 0.009 |
| Zirconia (ZrO²) | 0.066 | 0.061 | 0.060 | 0.015 | 0.009 | 0.009 | 0.013 | 0.009 | 0.009 |
| FR-4 | 0.016 | 0.015 | 0.015 | 0.004 | 0.002 | 0.002 | 0.004 | 0.002 | 0.002 |
| PDA(Polydopamine) | 0.011 | 0.010 | 0.009 | 0.002 | 0.001 | 0.001 | 0.002 | 0.001 | 0.001 |
| PFA (Perfluoroalkoxy) | 0.010 | 0.009 | 0.009 | 0.002 | 0.001 | 0.001 | 0.002 | 0.001 | 0.001 |
| PVDF | 0.015 | 0.014 | 0.013 | 0.004 | 0.002 | 0.002 | 0.004 | 0.002 | 0.002 |
| (Polyvinylidene fluoride) | | | | | | | | | |
| PFDA | 0.010 | 0.009 | 0.009 | 0.002 | 0.001 | 0.001 | 0.002 | 0.001 | 0.001 |
| (Perfluorodecanoic acid) | | | | | | | | | |
| PTFE | 0.010 | 0.009 | 0.009 | 0.002 | 0.001 | 0.001 | 0.002 | 0.001 | 0.001 |
| (Polytetrafluoroethylene) | | | | | | | | | |

An ultrasound wave having penetrated through the first unit 321, the second unit 322, the inductive body 323-1, the air layer, and the electrified body 323-2 may be reflected by the support 323-3 with very high reflectivity. The reflected ultrasound wave may induce additional vibrations within the triboelectric power harvesting unit 300. Additional vibrations may further increase energy efficiency of the triboelectric power harvesting unit 300.

Referring to Table 1 and FIG. 9, the inductive body 321-1 of the first unit 321 is in contact with the first silicon layer 310, and thus, may include barium titanate (BaTiO₃) having a highest transmittance with respect to silicon. In addition, the supports 321-3 and 322-3 in the first unit 321 and the second unit 322 may include alumina (Al₂O₃) and/or zirconia (ZrO₂) having relatively high transmittance with respect to gold (Au), and the support 323-2 of the third unit 323 may include a fluorine compound (e.g., PFA, FTFE, PFDA, or PDA) having a relatively low transmittance with respect to gold (Au). A power generation amount of the triboelectric power harvesting unit 300 may be improved by configuring a material of each structure in consideration of a transmittance and an amount of reflection of an ultrasound wave.

Although the present disclosure has been described with reference to an embodiment illustrated in the drawings, this is only an example, and it will be understood by those of ordinary skill in the art that various changes in the form and details may be made therein without departing from the spirit and scope of the present disclosure.
This research patent was supported by the Ministry of SMEs and Startups and the Korea Startup Promotion Agency through the DIPS 1000+ program (20241755) as part of the Super Gap Startup Development Project.
This research patent was supported by the 2022 research fund from the Ministry of Science and ICT and the National Research Foundation of Korea (NRF) under the Electronic Medicine Technology Development Program (2022M3E5E9016662).
This research patent was supported by the 2023 research fund from the Ministry of Trade, Industry and Energy and the Korea Evaluation Institute of Industrial Technology (KEIT) under the Next-Generation Intelligent Semiconductor Technology Development Program (20025736).

1. A system having an electroceutical for stimulating a nerve, the system comprising:
the electroceutical configured to sense a signal generating from the nerve and provide stimulation to the nerve; and
an electronic device configured to control the electroceutical,
wherein the electroceutical comprises:
a plurality of lead wires disposed to surround the nerve;
a header connector comprising a plurality of pins connected to the plurality of lead wires; and
a microcontroller unit (MCU) configured to change an electrical operation of the header connector, based on a control signal received from the electronic device, wherein the plurality of pins comprises:
one ground (GND) pin; and
a plurality of general-purpose input/output (GPIO) pins, wherein the MCU is configured to control an electrical operation of an individual GPIO pin of the plurality of GPIO pins,
wherein the plurality of GPIO pins are connected to different neural regions, respectively, and each of the plurality of GPIO pins is configured to receive a signal generated from the nerve or outputs a pulse signal according to a control by the MCU to provide the stimulation to the nerve,
wherein the MCU is further configured to:
monitor a battery of the electroceutical;
sense an analog signal generated from the nerve; and
provide the stimulation to the nerve, and
wherein the MCU is further configured to set an electrical state of the plurality of GPIO pin based on a timer interrupt generated in the MCU.

2-4. (canceled)

5. The system of claim 1, wherein the electroceutical comprises:
a housing comprising a plurality of regions;
a triboelectric power harvesting unit configured to generate energy based on an ultrasound wave provided from outside of the electroceutical;
a plurality of antennas operating in different frequency bands; and
shielding metal configured to shield electromagnetic interference caused by at least one of the plurality of antennas, and
the electroceutical receives a control signal from the electronic device through an antenna having an operating frequency of 2.4 GHz among the plurality of antennas.

6. The system of claim 5, wherein the electronic device displays an interface for controlling the electroceutical,
wherein the interface is configured to:
set an electrical operating mode of each of the plurality of GPIO pins, and
wherein the electrical operation mode comprising a sensing mode configured to receive the signal generated from the nerve, a stimulation mode configured to provide the pulse signal to the nerve, and a disable mode configured to be disabled.

7. The system of claim 6, wherein the interface is further configured to:
set a center frequency of a pulse signal generated for nerve stimulation; and
set a pulse width of the pulse signal.

8. The system of claim 7, wherein the interface is further configured to:
set whether to apply frequency random modulation to the pulse signal and set a range of the frequency random modulation with respect to the pulse signal; and
set a ratio between the center frequency and a variable frequency in which the frequency random modulation is applied to the pulse signal.

9. The system of claim 5, wherein the triboelectric power harvesting unit comprises:
a power generating unit;
a first silicon layer arranged between an upper surface of the power generating unit and an inner surface of the electroceutical;
a second silicon layer arranged on a lower surface of the power generating unit; and
a device housing arranged to surround outer peripheries of the power generating unit, the first silicon layer, and the second silicon layer.

10. The system of claim 9, wherein the ultrasound waves provided from the outside penetrates through a titanium region from the plurality of regions and the first silicon layer to be introduced into the power generating unit, is reflected within the power generating unit, and causes repetitive vibrations.

11. The system of claim 1, wherein the electroceutical monitors a voltage of the battery and transmits the monitored voltage to the electronic device, and
the electronic device calculates a remaining capacity of the battery based on the received voltage, provides the remaining capacity through an interface, and displays a charging alarm of the electroceutical when the remaining capacity is equal to or less than a threshold value.
